(19) United States
(12) Patent Application Publication
(10) Pub. No.: US 2023/0146072 A1
(43) Pub. Date: May 11, 2023

(54) ANTI-GUCY2C ANTIBODIES AND USES THEREOF

(71) Applicant: PFIZER INC., NEW YORK, NY (US)

(72) Inventors: Chew Shun CHANG, Quincy, MA (US); Divya MATHUR, Scarsdale, NY (US); Adam Reid ROOT, Newbury, MA (US)

(73) Assignee: PFIZER INC., NEW YORK, NY (US)

(21) Appl. No.: 17/917,886

(22) PCT Filed: Oct. 14, 2021
(86) PCT No.: PCT/IB2021/052817

§ 371 Date: Oct. 7, 2022

Related U.S. Application Data
(60) Provisional application No. 63/007,073, filed on Apr. 8, 2020.

(51) Int. Cl. C07K 16/40; G01N 33/574

(52) U.S. Cl. CPC C07K 16/40; G01N 33/574; C07K 2317/565; C07K 2317/24; C07K 2317/33

(57) ABSTRACT

The present invention is directed to antibodies that specifically bind to GUCY2c and methods of using such antibodies in the diagnosis and/or treatment of cancer.

### FIELD

The present invention is directed to antibodies that bind GUCY2c (Guanylyl cyclase C). The invention further relates to compositions comprising antibodies to GUCY2c, and methods of using anti-GUCY2c antibodies as a diagnostic or medicament. Certain embodiments relate to methods of using anti-GUCY2c antibodies for the treatment, prevention and/or diagnosis of various diseases, including hyperproliferative disease, such as cancer.

### BACKGROUND

Cancer is a leading cause of death worldwide, accounting for more than 7 million deaths each year. Cancer mortality is nearly universally related to the spread of primary tumors to distant sites forming metastases and ultimately leading to death. This is particularly true for gastrointestinal cancer, including adenocarcinoma of the esophagus, stomach, colon, and rectum. Colorectal cancer (CRC) remains the fourth most diagnosed cancer, and the second leading cause of cancer death in the United States (Siegel R L, Miller K D, Jemal A. Cancer statistics, 2016. CA Cancer J Clin., 66:7-30, 2016). Worldwide, colorectal cancer accounts for as many as 1.2 million new cases and 600,000 deaths per year (Brenner H, Kloor M, Pox C P. Colorectal cancer. Lancet, 383:1490-502, 2014).

Guanylyl cyclase C (GUCY2c) (also known as STAR, ST Receptor, GUC2C, GUCY2C, GC-C and GCC) is a transmembrane cell surface receptor that functions in the maintenance of intestinal fluid, electrolyte homeostasis and cell proliferation (Carrithers et al., Proc Natl Acad Sci USA 100: 3018-3020, 2003; Mann et al., Biochem Biophys Res Commun 239: 463-466, 1997; Pitari et al., Proc Natl Acad Sci USA 100: 2695-2699, 2003); GenBank Accession No. NM.sub.-004963, and GenPept Accession No. NP-004954). This function is mediated through binding of guanylin (Wiegand et al. FEBS Lett. 311:150-154, 1992) and uroguanylin (Hamra et al. Proc Natl Acad Sci USA 9(22):10464-10468, 1993). GUCY2c also is a receptor for heat-stable enterotoxin (ST) which is a peptide produced by E. coli, as well as other infectious organisms (Rao, M. C. Ciba Found. Symp. 112:74-93, 1985; Knoop F. C. and Owens, M. J. Pharmacol. Toxicol. Methods 28:67-72, 1992). Binding of ST to GUCY2c activates a signal cascade that results in enteric disease, e.g., diarrhea.

GUCY2c has been characterized as a protein involved in cancers, including colorectal cancer, pancreatic cancer, gastric cancer, hepatic cancer, and esophageal cancer (Carrithers et al., Dis Colon Rectum 39:171-181, 1996; Buc et al. Eur J Cancer 41: 1618-1627, 2005; Carrithers et al., Gastroenterology 107: 1653-1661, 1994; Urbanski et al., Biochem Biophys Acta 1245: 29-36, 1995).

As a cell surface protein, GUCY2c can serve as a therapeutic target for receptor binding proteins such as antibodies or ligands. GUCY2c is expressed on the apical side of epithelial cells lining the mucosa of the small intestine, large intestine and rectum (Carrithers et al., Dis Colon Rectum 39: 171-181, 1996). GUCY2c expression is maintained upon neoplastic transformation of intestinal epithelial cells, with expression in all primary and metastatic colorectal tumors (Carrithers et al., 1996; Buc et al.; Carrithers et al., 1994). GUCY2c expression has also been detected in esophageal cells diagnosed as Barrett's esophagus, esophageal cancer and gastric cancer.

There remains a need for molecules and/or compositions which can specifically target and specifically bind to primary and metastatic colorectal cancer cells. There is a need for improved methods of diagnosing individuals who are suspected of suffering from colorectal cancer, especially individuals who are suspected of suffering from metastasis of colorectal cancer cells.

### SUMMARY

It is demonstrated that certain anti-GUCY2c antibodies are effective in vivo to diagnose, prevent and/or treat cancer. The invention disclosed herein is directed to antibodies that specifically bind to GUCY2c. In some embodiments, the antibody can be, for example, a human, humanized, or chimeric antibody. In some embodiments, the anti-GUCY2c antibody is a chimeric antibody having rabbit constant regions.

In one aspect, the invention provides an isolated antibody which specifically binds to GUCY2c, wherein the antibody comprises a heavy chain variable region (VH) comprising a VH complementarity determining region one (CDR1), VH CDR2, and VH CDR3 of the amino acid sequence shown in SEQ ID NO: 2; and a light chain variable region (VL) comprising a VL CDR1, VL CDR2, and VL CDR3 of the amino acid sequence shown in SEQ ID NO: 1.

In some embodiments, the VH region comprises the amino acid sequence shown in SEQ ID NO: 2, or a variant with one or several conservative amino acid substitutions in residues that are not within a CDR and/or the VL region comprises the amino acid sequence shown in SEQ ID NO: 1, or a variant thereof with one or several amino acid substitutions in amino acids that are not within a CDR. In some embodiments, the antibody comprises a light chain comprising the sequence shown in SEQ ID NO: 11, 14, 15, 16, 17, 18, or 19 and/or a heavy chain comprising the sequence shown in SEQ ID NO: 9, 10, 12 or 13.

In another aspect, the invention provides an isolated antibody which specifically binds to GUCY2c, wherein the antibody comprises a VH CDR1 comprising the amino acid sequence of SEQ ID NO: 6, a VH CDR2 comprising the amino acid sequence of SEQ ID NO: 7, a VH CDR3 comprising the amino acid sequence shown in SEQ ID NO: 8, a VL CDR1 comprising the amino acid sequence shown in SEQ ID NO: 3, a VL CDR2 comprising the amino acid sequence shown in SEQ ID NO: 4 and a VL CDR3 comprising the amino acid sequence shown in SEQ ID NO: 5.

In another aspect, the invention provides an isolated antibody which competes for binding to GUCY2c with any one of the preceding antibodies.

In some embodiments, the antibody can be a human antibody, a rabbit antibody, a humanized antibody, a rabbitized antibody, or a chimeric antibody. In some embodiments, the antibody is a chimeric rabbit antibody.

In some embodiments, the antibody comprises a constant region. In some embodiments, the antibody is a rabbit IgA, IgE, IgG or IgM antibody. In some embodiments, the antibody comprises a rabbit kappa light chain. In other embodiments, the antibody comprises a rabbit lambda light chain. In other embodiments, the antibody is of the human IgG₁, IgG₂, IgG_{2Δa}, IgG₃, IgG₄, IgG_{4Δb}, IgG_{4Δc}, IgG₄S228P, IgG_{4Δb} S228P, or IgG_{4Δc} S228P subclass.

In another aspect, the invention provides an isolated antibody which specifically binds to GUCY2c and competes with and/or binds to the same GUCY2c epitope as the antibodies as described herein.

In another aspect, the invention provides an isolated polynucleotide comprising a nucleotide sequence encoding a GUCY2c antibody as described herein. In another aspect, the invention provides a vector comprising the polynucleotide.

In another aspect, the invention provides an isolated host cell that recombinantly produces a GUCY2c antibody as described herein.

In another aspect, the invention provides a method of producing an anti-GUCY2c antibody, the method comprising: culturing a cell line that recombinantly produces the antibody as described herein under conditions wherein the antibody is produced; and recovering the antibody.

In another aspect, the invention provides a method of producing an anti-GUCY2c antibody, the method comprising: culturing a cell line comprising nucleic acid encoding an antibody comprising a heavy chain comprising the amino acid sequence shown in SEQ ID NO: 9, 10, 12 or 13 and a light chain comprising the amino acid sequence shown in SEQ ID NO: 11, 14, 15, 16, 17, 18, or 19 under conditions wherein the antibody is produced; and recovering the antibody.

In some embodiments, the antibodies of the present invention may be detectably labeled, attached to a solid support, or the like.

In some embodiments, the heavy and light chains are encoded on separate vectors. In other embodiments, heavy and light chains are encoded on the same vector.

Also provided is the use of any of the anti-GUCY2c antibodies provided herein for the diagnoisis of cancer or for inhibiting tumor growth or progression in a subject in need thereof. In some embodiments, the anti-GUCY2c antibody reduces weight gain in the subject.

Also provided are anti-GUCY2c antibodies for use in the diagnosis of a cancer. In some embodiments, the cancer is, for example without limitation, gastric cancer, sarcoma, lymphoma, Hodgkin's lymphoma, leukemia, head and neck cancer, thymic cancer, epithelial cancer, salivary cancer, liver cancer, stomach cancer, thyroid cancer, lung cancer (including, for example, non-small-cell lung carcinoma), ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma.

In one aspect, the invention provides a method for diagnosing cancer in a subject, the method comprising contacting a test sample of tissue cells suspected of containing cancerous tumor cells obtained from the subject with the antibody of the present invention.

In some embodiments, the method further comprises detecting the formation of a complex between the antibody and GUCY2c in the sample, and classifying a higher level of formation of such a complex in the test sample as compared to the level of formation of such a complex in a control sample of normal tissue cells from the same type of tissue as the sample, as diagnostic of the presence of cancer in the subject.

In some embodiments, the cancer is selected from the group consisting of colorectal cancer, gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma.

In some embodiments, the cancer is gastric cancer.

In some embodiments, the antibody is detectably labeled.

In another aspect, the invention provides a method of diagnosing the presence of cancer in a subject, the method comprising determining the level of expression of—Guanylyl cyclase C (GUCY2c) in a test sample of tissue cells obtained from tissue suspected of containing cancerous tumor cells in the subject and in a control sample of known normal cells obtained from the same type of tissue as the test sample, wherein determining the level of expression of GUCY2c comprises employing an antibody of the present invention, and classifying a higher level of expression of GUCY2c in the test sample as compared to the control sample, as diagnostic of the presence of cancer in the subject from which the test sample was obtained.

In some embodiments, the step employing the antibody comprises an immunohistochemistry or Western blot analysis.

In some embodiments, the invention concerns a composition of matter comprising an anti-GUCY2c antibody as described herein, a chimeric anti-GUCY2c antibody as described herein, or a rabbitized anti-GUCY2c antibody as described herein, in combination with a carrier. Optionally, the carrier is a pharmaceutically acceptable carrier.

In some embodiments, the invention concerns an article of manufacture comprising a container and a composition of matter contained within the container, wherein the composition of matter comprise an anti-GUCY2c antibody as described herein, a chimeric anti-GUCY2c antibody as described herein, or a rabbitized anti-GUCY2c antibody as described herein. The article may further optionally comprise a label affixed to the container, or a package insert included with the container, that refers to the use of the composition of matter for the therapeutic treatment or diagnostic detection of a tumor. Another embodiment of the present invention is directed to the use of an anti-GUCY2c antibody as described herein, a chimeric anti-GUCY2c antibody as described herein, or a a rabbitized anti-GUCY2c antibody as described herein, for the preparation of a medicament useful for the treatment of a condition which is responsive to the anti-GUCY2c antibody.

### DETAILED DESCRIPTION

Disclosed herein are antibodies that specifically bind to GUCY2c. Methods of making anti-GUCY2c antibodies, compositions comprising these antibodies, and methods of using these antibodies as a diagnostic and/or medicament are provided. The anti-GUCY2c antibodies described herein can be used to detect the presence of GUCY2c in a sample, and/or the prevention and/or treatment of cancer and/or other diseases.

General Techniques The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J.E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

The following terms, unless otherwise indicated, shall be understood to have the following meanings: the term “isolated molecule” as referring to a molecule (where the molecule is, for example, a polypeptide, a polynucleotide, or an antibody) that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy-chain constant regions that correspond to the different classes of immunoglobulins are called alpha, delta, epsilon, gamma, and mu, respectively. The subunit structures and three-dimensional configurations of different classes of immunoglobulins are well known. A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art, a “constant region” of an antibody refers to the constant region of the antibody light chain or the constant region of the antibody heavy chain, either alone or in combination.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example. As used herein, “humanized” antibody refers to forms of non-human (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance. As used herein, “rabbitized” antibody refers to forms of non-rabbit (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-rabbit immunoglobulin. Preferably, rabbitized antibodies are rabbit immunoglobulins (recipient antibody) in which residues from a CDR of the recipient are replaced by residues from a CDR of a non-rabbit species (donor antibody) such as mouse, rat, or goat having the desired specificity, affinity, and capacity. The rabbitized antibody may comprise residues that are found neither in the recipient antibody nor in the imported CDR or framework sequences, but are included to further refine and optimize antibody performance.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “epitope” refers to that portion of a molecule capable of being recognized by and bound by an antibody at one or more of the antibody's antigen-binding regions. Epitopes often consist of a surface grouping of molecules such as amino acids or sugar side chains and have specific three-dimensional structural characteristics as well as specific charge characteristics. In some embodiments, the epitope can be a protein epitope. Protein epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds. The term “antigenic epitope” as used herein, is defined as a portion of an antigen to which an antibody can specifically bind as determined by any method well known in the art, for example, by conventional immunoassays. Once a desired epitope on an antigen is determined, it is possible to generate antibodies to that epitope, e.g., using the techniques described in the present specification. Alternatively, during the discovery process, the generation and characterization of antibodies may elucidate information about desirable epitopes. From this information, it is then possible to competitively screen antibodies for binding to the same epitope. An approach to achieve this is to conduct competition and cross-competition studies to find antibodies that compete or cross-compete with one another for binding to GUCY2c, e.g., the antibodies compete for binding to the antigen.

As used herein, “GUCY2c,” refers to mammalian guanylyl cyclase C (GUCY2c), preferably human GUCY2c protein. The term “GUCY2c” may be used interchangeably with the term “GUCY2C”. A nucleotide sequence for human GUCY2c is disclosed as GenBank Accession No. NM_004963, which is incorporated herein by reference. The amino acid sequence for human GUCY2c is disclosed as GenBank Accession No. NP_004954, which is incorporated herein by reference.

Typically, a naturally occurring allelic variant has an amino acid sequence at least 95%, 97% or 99% identical to the protein described in GenBank Accession No. NP_004954. The GUCY2c protein is characterized as a transmembrane cell surface receptor protein, and is believed to play a critical role in the maintenance of intestinal fluid, electrolyte homeostasis and cell proliferation.

As used herein, an “antibody that binds to GUCY2c,” an “antibody that recognizes GUCY2c,” an “anti-GUCY2c antibody,” an “anti-GUCY2c antibody molecule” or a “GUCY2c antibody” comprises a molecule which combines at least one binding domain of an antibody (as herein defined) with at least one binding domain of an anti-GUCY2c antibody (as defined in this application). The GUCY2c antibody molecule of the present invention includes antibodies and antigen-binding fragments thereof that interact with or recognize, e.g., bind (e.g., bind specifically) to GUCY2c, e.g., human GUCY2c, mouse GUCY2c, rat GUCY2c, cynomolgus GUCY2c.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (-O-) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, an antibody “interacts with” GUCY2c when the equilibrium dissociation constant is equal to or less than 20 nM, preferably less than about 6 nM, more preferably less than about 1 nM, most preferably less than about 0.2 nM, as measured by the methods disclosed herein in Example 7.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a GUCY2c epitope is an antibody that binds this epitope with greater affinity, avidity, more readily, and/or with greater duration than it binds to other GUCY2c epitopes or non-GUCY2c epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

A “functional Fc region” possesses at least one effector function of a native sequence Fc region. Exemplary “effector functions” include C1q binding; complement dependent cytotoxicity; Fc receptor binding; antibody-dependent cell-mediated cytotoxicity; phagocytosis; down-regulation of cell surface receptors (e.g. B cell receptor), etc. Such effector functions generally require the Fc region to be combined with a binding domain (e.g. an antibody variable domain) and can be assessed using various assays known in the art for evaluating such antibody effector functions.

As used herein, “treatment” is an approach for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of a tumor, remission of cancer, decreasing symptoms resulting from cancer, increasing the quality of life of those suffering from cancer, decreasing the dose of other medications required to treat cancer, delaying the progression of cancer, curing a cancer, and/or prolong survival of patients having cancer.

“Ameliorating” means a lessening or improvement of one or more symptoms as compared to not administering an anti-GUCY2c antibody. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. In more specific aspects, an effective amount prevents, alleviates or ameliorates symptoms of disease, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more symptoms of a disease such as, for example, cancer including, for example without limitation, gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma, decreasing the dose of other medications required to treat the disease, enhancing the effect of another medication, and/or delaying the progression of the cancer in patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

An “individual” or a “subject” is a mammal, more preferably, a human. Mammals also include, but are not limited to, farm animals (e.g., cows, pigs, horses, chickens, etc.), sport animals, pets, primates, horses, dogs, cats, mice and rats.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “kₒₙ”, as used herein, refers to the rate constant for association of an antibody to an antigen. Specifically, the rate constants (kₒₙ and k_{off}) and equilibrium dissociation constants are measured using full-length antibodies and/or Fab antibody fragments (i.e. univalent) and GUCY2c.

The term “k_{off}”, as used herein, refers to the rate constant for dissociation of an antibody from the antibody/antigen complex.

The term “K_{D}”, as used herein, refers to the equilibrium dissociation constant of an antibody-antigen interaction.

Reference to “about” a value or parameter herein includes (and describes) embodiments that are directed to that value or parameter per se. For example, description referring to “about X” includes description of “X.” Numeric ranges are

The term “intradermal administration,” or “administered intradermally,” in the context of administering a substance to a mammal including a human, refers to the delivery of the substance into the dermis layer of the skin of the mammal. The skin of a mammal is composed of an epidermis layer, a dermis layer, and a subcutaneous layer. The epidermis is the outer layer of the skin. The dermis, which is the middle layer of the skin, contains nerve endings, sweat glands and oil (sebaceous) glands, hair follicles, and blood vessels. The subcutaneous layer is made up of fat and connective tissue that houses larger blood vessels and nerves. In contrast in intradermal administration, “subcutaneous administration” refers to the administration of a substance into the subcutaneous layer and “topical administration” refers to the administration of a substance onto the surface of the skin.

The term “neoplastic disorder” refers to a condition in which cells proliferate at an abnormally high and uncontrolled rate, the rate exceeding and uncoordinated with that of the surrounding normal tissues. It usually results in a solid lesion or lump known as “tumor.” This term encompasses benign and malignant neoplastic disorders. The term “malignant neoplastic disorder”, which is used interchangeably with the term “cancer” in the present disclosure, refers to a neoplastic disorder characterized by the ability of the tumor cells to spread to other locations in the body (known as “metastasis”). The term “benign neoplastic disorder” refers to a neoplastic disorder in which the tumor cells lack the ability to metastasize.

The term “preventing” or “prevent” refers to (a) keeping a disorder from occurring or (b) delaying the onset of a disorder or onset of symptoms of a disorder.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present invention. The materials, methods, and examples are illustrative only and not intended to be limiting.

### Anti-GUCY2c Antibodies

Provided herein are anti-GUCY2c antibodies. In some embodiments, the anti-GUCY2c antibodies specifically bind to human GUCY2c and cross-react with cynomolgus monkey GUCY2c, and do not cross-react with mouse GUCY2c. The antibodies useful in the present invention can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody portion (e.g., a domain antibody), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, the anti-GUCY2c antibody is a monoclonal antibody. In some embodiments, the antibody is a mouse antibody, chimeric rabbit antibody or rabbitized antibody. In some embodiments, the antibody is a human or humanized antibody.

The anti-GUCY2c antibodies may be made by any method known in the art. General techniques for production of human and mouse antibodies are known in the art and/or are described herein.

Anti-GUCY2c antibodies may be characterized using methods well known in the art. For example, one method is to identify the epitope to which it binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an anti-GUCY2c antibody binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with an anti-GUCY2c antibody. In another example, the epitope to which the anti-GUCY2c antibody binds can be determined in a systematic screening by using overlapping peptides derived from the GUCY2c sequence and determining binding by the anti-GUCY2c antibody. According to the gene fragment expression assays, the open reading frame encoding GUCY2c is fragmented either randomly or by specific genetic constructions and the reactivity of the expressed fragments of GUCY2c with the antibody to be tested is determined. The gene fragments may, for example, be produced by PCR and then transcribed and translated into protein in vitro, in the presence of radioactive amino acids. The binding of the antibody to the radioactively labeled GUCY2c fragments is then determined by immunoprecipitation and gel electrophoresis. Certain epitopes can also be identified by using large libraries of random peptide sequences displayed on the surface of phage particles (phage libraries) or yeast (yeast display). Alternatively, a defined library of overlapping peptide fragments can be tested for binding to the test antibody in simple binding assays. In an additional example, mutagenesis of an antigen, domain swapping experiments and alanine scanning mutagenesis can be performed to identify residues required, sufficient, and/or necessary for epitope binding. For example, alanine scanning mutagenesis experiments can be performed using a mutant GUCY2c in which various residues of the GUCY2c polypeptide have been replaced with alanine. By assessing binding of the antibody to the mutant GUCY2c, the importance of the particular GUCY2c residues to antibody binding can be assessed.

Yet another method which can be used to characterize an anti-GUCY2c antibody is to use competition assays with other antibodies known to bind to the same antigen, i.e., various fragments of GUCY2c, to determine if the anti-GUCY2c antibody binds to the same epitope as other antibodies. Competition assays are well known to those of skill in the art, including in an ELISA format.

Accordingly, the invention provides any of the following, or compositions (including pharmaceutical compositions) comprising an antibody having a partial light chain sequence and a partial heavy chain sequence as found in Table 1, or variants thereof. In Table 1, the underlined sequences are CDR sequences.

TABLE 1
Variable Regions Sequences of
Anti-GUCY2c antibodies

| mAb | Light Chain | Heavy Chain |
|---|---|---|
| Ab288 | DIVLTQSPASLAVSLGQRAT | DVQLQESGPGLVKPSQSLSLT |
| | ISCRASESVEYFGTSFMQW | CTVTGYSITSDYAWNWIRQFP |
| | YQQRPGQPPKLLIYAASNV | GNNLEWMGYISYSGSTRYNPS |
| | ESGVPVRFSGSGSGTDFSL | LKSRISITRDTSKNQFFLQLN |
| | NIHPVEEDDIAMYFCQQSR | SVTSEDTATYYCAREDGYVAM |
| | KVPWTFGGGTNLEIK | DYWGQGTSVTVSS |
| | (SEQ ID NO: 1) | (SEQ ID NO: 2) |

The invention also provides CDR portions of antibodies to GUCY2c. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

In some embodiments, the antibody comprises three CDRs of any one of the heavy chain variable regions shown in Table 1. In some embodiments, the antibody comprises three CDRs of any one of the light chain variable regions shown in Table 1. In some embodiments, the antibody comprises three CDRs of any one of the heavy chain variable regions shown in Table 1, and three CDRs of any one of the light chain variable regions shown in Table 1.

Table 2 provides examples of CDR sequences of anti-GUCY2c antibodies provided herein.

TABLE 2
Anti-GUCY2c antibodies (mAbs) and
their antigen-binding CDR sequences

| mAb | Chain | CDR1 | CDR2 | CDR3 |
|---|---|---|---|---|
| Ab288 | light | RASES | AASNV | QQSRK |
| | | VEYFG | ES | VPWT |
| | | TSFM | (SEQ ID | (SEQ ID |
| | | Q | NO: 4) | NO: 5) |
| | | (SEQ ID | | |
| | | NO: 3) | | |
| | heavy | SDYAW | YISYS | EDGYV |
| | | N | GSTRY | AMDY |
| | | (SEQ ID | NPSL | (SEQ ID |
| | | NO: 6) | KS | NO: 8) |
| | | | (SEQ ID | |
| | | | NO: 7) | |

In some embodiments, the antibody comprises the full-length heavy chain, with or without the C-terminal lysine, and/or the full-length light chain of anti-GUCY2c antibody Ab288. The amino acid sequence of Ab288 full-length heavy chain (SEQ ID NO: 9) is shown below (with the variable region underlined):

(SEQ ID NO: 9)
DVQLQESGPGLVKPSQSLSLTCTVTGYSITSDYAW
NWIRQFPGNNLEWMGYISYSGSTRYNPSLKSRISI
TRDTSKNQFFLQLNSVTSEDTATYYCAREDGYVAM
DYWGQGTSVTVSSAKTTPPSVYPLAPGSAAQTNSM
VTLGCLVKGYFPEPVTVTWNSGSLSSGVHTFPAVL
QSDLYTLSSSVTVPSSTWPSETVTCNVAHPASSTK
VDKKIVPRDCGCKPCICTVPEVSSVFIFPPKPKDV
LTITLTPKVTCVVVVDISKDDPEVQFSWFVDDVEV
HTAQTQPREEQFNSTFRSVSELPIMHQDWLNGKEF
KCRVNSAAFPAPIEKTISKTKGRPKAPQVYTIPPP
KEQMAKDKVSLTCMITDFFPEDITVEWQWNGQPAE
NYKNTQPIMDTDGSYFVYSKLNVQKSNWEAGNTFT
CSVLHEGLHNHHTEKSLSHSPGK

The amino acid sequence of Ab288 full-length heavy chain without the C-terminal lysine (SEQ ID NO: 10) is shown below (with the variable region underlined):

(SEQ ID NO: 10)
DVQLQESGPGLVKPSQSLSLTCTVTGYSITSDYAWNWIRQ
FPGNNLEWMGYISYSGSTRYNPSLKSRISITRDTSKNQFF
LQLNSVTSEDTATYYCAREDGYVAMDYWGQGTSVTVSSAK
TTPPSVYPLAPGSAAQTNSMVTLGCLVKGYFPEPVTVTWN
SGSLSSGVHTFPAVLQSDLYTLSSSVTVPSSTWPSETVTC
NVAHPASSTKVDKKIVPRDCGCKPCICTVPEVSSVFIFPP
KPKDVLTITLTPKVTCVVVDISKDDPEVQFSWFVDDVEVH
TAQTQPREEQFNSTFRSVSELPIMHQDWLNGKEFKCRVNS
AAFPAPIEKTISKTKGRPKAPQVYTIPPPKEQMAKDKVSL
TCMITDFFPEDITVEWQWNGQPAENYKNTQPIMDTDGSYF
VYSKLNVQKSNWEAGNTFTCSVLHEGLHNHHTEKSLSHSP
G

The amino acid sequence of Ab288 full-length light chain (SEQ ID NO: 11) is shown below (with the variable region underlined):

(SEQ ID NO: 11)
DIVLTQSPASLAVSLGQRATISCRASESVEYFGTSFMQWY
QQRPGQPPKLLIYAASNVESGVPVRFSGSGSGTDFSLNI
HPVEEDDIAMYFCQQSRKVPWTFGGGTNLEIKRTDAAPTV
SIFPPSSEQLTSGGASVVCFLNNFYPKDINVKWKIDGSER
QNGVLNSWTDQDSKDSTYSMSSTLTLTKDEYERHNSYTCE
ATHKTSTSPIVKSFNRNEC

In some embodiments, the antibody comprises the full-length heavy chain, with or without the C-terminal lysine, and/or the full-length light chain of anti-GUCY2c antibody Ab288R. The amino acid sequence of Ab288R full-length heavy chain (SEQ ID NO: 12) is shown below (with the variable region underlined):

(SEQ ID NO: 12)
DVQLQESGPGLVKPSQSLSLTCTVTGYSITSDYAWNWIRQ
FPGNNLEWMGYISYSGSTRYNPSLKSRISITRDTSKNQFF
LQLNSVTSEDTATYYCAREDGYVAMDYWGQGTSVTVSSGQ
PKAPSVFPLAPCCGDTPSSTVTLGCLVKGYLPEPVTVTWN
SGTLTNGVRTFPSVRQSSGLYSLSSVVSVTSSSQPVTCNV
AHPATNTKVDKTVAPSTCSKPTCPPPELLGGPSVFIFPPK
PKDTLMISRTPEVTCVVVDVSQDDPEVQFTWYINNEQVRT
ARPPLREQQFNSTIRVVSTLPIAHQDWLRGKEFKCKVHNK
ALPAPIEKTISKARGQPLEPKVYTMGPPREELSSRSVSLT
CMINGFYPSDISVEWEKNGKAEDNYKTTPAVLDSDGSYFL
YSKLSVPTSEWQRGDVFTCSVMHEALHNHYTQKSISRSPG
K

The amino acid sequence of Ab288R full-length heavy chain without the C-terminal lysine (SEQ ID NO: 13) is shown below (with the variable region underlined):

(SEQ ID NO: 13)
DVQLQESGPGLVKPSQSLSLTCTVTGYSITSDYAWNWIRQ
FPGNNLEWMGYISYSGSTRYNPSLKSRISITRDTSKNQFF
LQLNSVTSEDTATYYCAREDGYVAMDYWGQGTSVTVSSGQ
PKAPSVFPLAPCCGDTPSSTVTLGCLVKGYLPEPVTVTWN
SGTLTNGVRTFPSVRQSSGLYSLSSVVSVTSSSQPVTCNV
AHPATNTKVDKTVAPSTCSKPTCPPPELLGGPSVFIFPPK
PKDTLMISRTPEVTCVVVDVSQDDPEVQFTWYINNEQVRT
ARPPLREQQFNSTIRVVSTLPIAHQDWLRGKEFKCKVHNK
ALPAPIEKTISKARGQPLEPKVYTMGPPREELSSRSVSLT
CMINGFYPSDISVEWEKNGKAEDNYKTTPAVLDSDGSYFL
YSKLSVPTSEWQRGDVFTCSVMHEALHNHYTQKSISRSPG

The amino acid sequence of Ab288R full-length light chain (SEQ ID NO: 14) is shown below (with the variable region underlined):

(SEQ ID NO: 14)
DIVLTQSPASLAVSLGQRATISCRASESVEYFGTSFMQWY
QQRPGQPPKLLIYAASNVESGVPVRFSGSGSGTDFSLNIH
PVEEDDIAMYFCQQSRKVPWTFGGGTNLEIKGDPVAPTVL
IFPPAADQVATGTVTIVCVANKYFPDVTVTWEVDGTTQTT
GIENSKTPQNSADCTYNLSSTLTLTSTQYNSHKEYTCRVT
QGTTSVVQSFNRGDC

The amino acid sequence of Ab288R full-length light chain without natural extra cys for disulfide bonding with FW3 of rabbit VL (SEQ ID NO: 15) is shown below (with the variable region underlined):

(SEQ ID NO: 15)
DIVLTQSPASLAVSLGQRATISCRASESVEYFGTSFMQWY
QQRPGQPPKLLIYAASNVESGVPVRFSGSGSGTDFSLNIH
PVEEDDIAMYFCQQSRKVPWTFGGGTNLEIKGDPVAPTVL
IFPPAADQVATGTVTIVCVANKYFPDVTVTWEVDGTTQTT
GIENSKTPQNSADXTYNLSSTLTLTSTQYNSHKEYTCRVT
QGTTSVVQSFNRGDC,
wherein X is A, D, E, F, G, H, I, K, L,
M, N, P, Q, R, S, T, V, W, or Y.

The amino acid sequence of Ab288R full-length light chain with the natural extra cys substituted with ser (SEQ ID NO: 16) is shown below (with the variable region underlined):

(SEQ ID NO: 16)
DIVLTQSPASLAVSLGQRATISCRASESVEYFGTSFMQWY
QQRPGQPPKLLIYAASNVESGVPVRFSGSGSGTDFSLNIH
PVEEDDIAMYFCQQSRKVPWTFGGGTNLEIKGDPVAPTVL
IFPPAADQVATGTVTIVCVANKYFPDVTVTWEVDGTTQTT
GIENSKTPQNSADSTYNLSSTLTLTSTQYNSHKEYTCRVT
QGTTSVVQSFNRGDC

The amino acid sequence of Ab288R full-length light chain with a lysine substitution (bold) (SEQ ID NO: 17) is shown below (with the variable region underlined):

(SEQ ID NO: 17)
DIVLTQSPASLAVSLGQRATISCRASESVEYFGTSFMQWY
QQRPGQPPKLLIYAASNVESGVPVRFSGSGSGTDFSLNIH
PVEEDDIAMYFCQQSRKVPWTFGGGTNLEIKGDPVAPTVL
IFPPAADQVATGTVTIVCVANKYFPDVTVTWEVDGTTQTT
GIENSKTPQNSADCTYNLSSTLTLTSTQYNSHKEYTCKVT
QGTTSWQSFNRGDC

The amino acid sequence of Ab288R full-length light chain without natural extra cys and with a lysine substitution (bold) (SEQ ID NO: 18) is shown below (with the variable region underlined):

(SEQ ID NO: 18)
DIVLTQSPASLAVSLGQRATISCRASESVEYFGTSFMQWY
QQRPGQPPKLLIYAASNVESGVPVRFSGSGSGTDFSLNIH
PVEEDDIAMYFCQQSRKVPWTFGGGTNLEIKGDPVAPTVL
IFPPAADQVATGTVTIVCVANKYFPDVTVTWEVDGTTQTT
GIENSKTPQNSADXTYNLSSTLTLTSTQYNSHKEYTCKVT
QGTTSWQSFNRGDC, wherein X is A, D,
E, F, G, H, I, K, L, M, N, P, Q,
R, S, T, V, W, or Y.

The amino acid sequence of Ab288R full-length light chain with the natural extra cys substituted with ser and a lysine substitution (bold) (SEQ ID NO: 19) is shown below (with the variable region underlined):

(SEQ ID NO: 19)
DIVLTQSPASLAVSLGQRATISCRASESVEYFGTSFMQWY
QQRPGQPPKLLIYAASNVESGVPVRFSGSGSGTDFSLNIH
PVEEDDIAMYFCQQSRKVPWTFGGGTNLEIKGDPVAPTVL
IFPPAADQVATGTVTIVCVANKYFPDVTVTWEVDGTTQTT
GIENSKTPQNSADSTYNLSSTLTLTSTQYNSHKEYTCKVT
QGTTSVVQSFNRGDC

The invention also provides methods of generating, selecting, and making anti-GUCY2c antibodies. The antibodies of this invention can be made by procedures known in the art. In some embodiments, antibodies may be made recombinantly and expressed using any method known in the art.

In some embodiments, antibodies may be prepared and selected by phage display technology. See, for example, U.S. Pat. Nos. 5,565,332; 5,580,717; 5,733,743; and 6,265,150; and Winter et al., Annu. Rev. Immunol. 12:433-455, 1994. Alternatively, the phage display technology (McCafferty et al., Nature 348:552-553, 1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S. and Chiswell, David J. Current Opinion in Structural Biology 3:564-571, 1993. Several sources of V-gene segments can be used for phage display. Clackson et al., Nature 352:624-628, 1991, isolated a diverse array of anti-oxazolone antibodies from a small random combinatorial library of V genes derived from the spleens of immunized mice. A repertoire of V genes from human donors can be constructed and antibodies to a diverse array of antigens (including self-antigens) can be isolated essentially following the techniques described by Mark et al., J. Mol. Biol. 222:581-597, 1991, or Griffith et al., EMBO J. 12:725-734, 1993. In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” (Marks et al., Bio/Technol. 10:779-783, 1992). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266, 1993. Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213). Unlike traditional humanization of rodent antibodies by CDR grafting, this technique provides completely human antibodies, which have no framework or CDR residues of rodent origin.

In some embodiments, antibodies may be made using hybridoma technology. It is contemplated that any mammalia subject including humans or antibody producing cells therefrom can be manipulated to serve as the basis for production of mammalian, including human, hybridoma cell lines. The route and schedule of immunization of the host animal are generally in keeping with established and conventional techniques for antibody stimulation and production, as further described herein. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., 1975, Nature 256:495-497 or as modified by Buck, D. W., et al., In Vitro, 18:377-381, 1982. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the GUCY2c monoclonal antibodies of the subject invention. The hybridomas or other immortalized B-cells are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for GUCY2c, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with a GUCY2c polypeptide, or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups, can yield a population of antibodies (e.g., monoclonal antibodies).

If desired, the anti-GUCY2c antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, the polynucleotide sequence may be used for genetic manipulation to “humanize” or “rabbitize” the antibody or to improve the affinity, or other characteristics of the antibody. Antibodies may also be customized for use, for example, in dogs, cats, primate, equines and bovines.

In some embodiments, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

Antibodies may be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. See, for example, Peeters, et al. Vaccine 19:2756, 2001; Lonberg, N. and D. Huszar Int. Rev. Immunol 13:65, 1995; and Pollock, et al., J Immunol Methods 231:147, 1999. Methods for making derivatives of antibodies, e.g., domain, single chain, etc. are known in the art.

Immunoassays and flow cytometry sorting techniques such as fluorescence activated cell sorting (FACS) can also be employed to isolate antibodies that are specific for GUCY2c.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA also may be modified, for example, by substituting the coding sequence for human heavy and light chain constant domains in place of the homologous murine sequences, Morrison et al., Proc. Nat. Acad. Sci. 81:6851, 1984, or by covalently joining to the immunoglobulin coding sequence all or part of the coding sequence for a non-immunoglobulin polypeptide. In that manner, “chimeric” or “hybrid” antibodies are prepared that have the binding specificity of a GUCY2c monoclonal antibody herein.

Antibody fragments can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

In some embodiments, a polynucleotide comprises a sequence encoding the heavy chain and/or the light chain variable regions of antibody Ab288. The sequence encoding the antibody of interest may be maintained in a vector in a host cell and the host cell can then be expanded and frozen for future use. Vectors (including expression vectors) and host cells are further described herein.

The invention includes affinity matured embodiments. For example, affinity matured antibodies can be produced by procedures known in the art (Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154(7):3310-9; Hawkins et al., 1992, J. Mol. Biol., 226:889-896; and PCT Publication No. W02004/058184).

The following methods may be used for adjusting the affinity of an antibody and for characterizing a CDR. One way of characterizing a CDR of an antibody and/or altering (such as improving) the binding affinity of a polypeptide, such as an antibody, termed “library scanning mutagenesis”. Generally, library scanning mutagenesis works as follows. One or more amino acid positions in the CDR are replaced with two or more (such as 3, 4, 5, 6, 7, 8, 9, 10, 11, 12, 13, 14, 15, 16, 17, 18, 19, or 20) amino acids using art recognized methods. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, e.g., about 20-80 clones (depending on the complexity of the library), from each library are screened for binding affinity to the target polypeptide (or other binding target), and candidates with increased, the same, decreased, or no binding are identified. Methods for determining binding affinity are well-known in the art. Binding affinity may be determined using, for example, Biacore™ surface plasmon resonance analysis, which detects differences in binding affinity of about 2-fold or greater, Kinexa® Biosensor, scintillation proximity assays, ELISA, ORIGEN® immunoassay, fluorescence quenching, fluorescence transfer, and/or yeast display. Binding affinity may also be screened using a suitable bioassay. Biacore™ is particularly useful when the starting antibody already binds with a relatively high affinity, for example a K_{D} of about 10 nM or lower.

In some embodiments, every amino acid position in a CDR is replaced (in some embodiments, one at a time) with all 20 natural amino acids using art recognized mutagenesis methods (some of which are described herein). This generates small libraries of clones (in some embodiments, one for every amino acid position that is analyzed), each with a complexity of 20 members (if all 20 amino acids are substituted at every position).

In some embodiments, the library to be screened comprises substitutions in two or more positions, which may be in the same CDR or in two or more CDRs. Thus, the library may comprise substitutions in two or more positions in one CDR. The library may comprise substitution in two or more positions in two or more CDRs. The library may comprise substitution in 3, 4, 5, or more positions, said positions found in two, three, four, five or six CDRs. The substitution may be prepared using low redundancy codons. See, e.g., Table 2 of Balint et al., 1993, Gene 137(1):109-18.

The CDR may be heavy chain variable region (VH) CDR3 and/or light chain variable region (VL) CDR3. The CDR may be one or more of VH CDR1, VH CDR2, VH CDR3, VL CDR1, VL CDR2, and/or VL CDR3. The CDR may be a Kabat CDR, a Chothia CDR, an extended CDR, an AbM CDR, a contact CDR, or a conformational CDR.

Candidates with improved binding may be sequenced, thereby identifying a CDR substitution mutant which results in improved affinity (also termed an “improved” substitution). Candidates that bind may also be sequenced, thereby identifying a CDR substitution which retains binding.

Multiple rounds of screening may be conducted. For example, candidates (each comprising an amino acid substitution at one or more position of one or more CDR) with improved binding are also useful for the design of a second library containing at least the original and substituted amino acid at each improved CDR position (i.e., amino acid position in the CDR at which a substitution mutant showed improved binding). Preparation, and screening or selection of this library is discussed further below.

Library scanning mutagenesis also provides a means for characterizing a CDR, in so far as the frequency of clones with improved binding, the same binding, decreased binding or no binding also provide information relating to the importance of each amino acid position for the stability of the antibody-antigen complex. For example, if a position of the CDR retains binding when changed to all 20 amino acids, that position is identified as a position that is unlikely to be required for antigen binding. Conversely, if a position of CDR retains binding in only a small percentage of substitutions, that position is identified as a position that is important to CDR function. Thus, the library scanning mutagenesis methods generate information regarding positions in the CDRs that can be changed to many different amino acids (including all 20 amino acids), and positions in the CDRs which cannot be changed or which can only be changed to a few amino acids.

Candidates with improved affinity may be combined in a second library, which includes the improved amino acid, the original amino acid at that position, and may further include additional substitutions at that position, depending on the complexity of the library that is desired, or permitted using the desired screening or selection method. In addition, if desired, adjacent amino acid position can be randomized to at least two or more amino acids. Randomization of adjacent amino acids may permit additional conformational flexibility in the mutant CDR, which may in turn, permit or facilitate the introduction of a larger number of improving mutations. The library may also comprise substitution at positions that did not show improved affinity in the first round of screening.

The second library is screened or selected for library members with improved and/or altered binding affinity using any method known in the art, including screening using Kinexa™ biosensor analysis, and selection using any method known in the art for selection, including phage display, yeast display, and ribosome display.

To express the anti-GUCY2c antibodies of the present invention, DNA fragments encoding VH and VL regions can first be obtained using any of the methods described above. Various modifications, e.g. mutations, deletions, and/or additions can also be introduced into the DNA sequences using standard methods known to those of skill in the art. For example, mutagenesis can be carried out using standard methods, such as PCR-mediated mutagenesis, in which the mutated nucleotides are incorporated into the PCR primers such that the PCR product contains the desired mutations or site-directed mutagenesis.

The invention encompasses modifications to the variable regions shown in Table 1 and the CDRs shown in Table 2. For example, the invention includes antibodies comprising functionally equivalent variable regions and CDRs which do not significantly affect their properties as well as variants which have enhanced or decreased activity and/or affinity. For example, the amino acid sequence may be mutated to obtain an antibody with the desired binding affinity to GUCY2c. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but framework alterations are also contemplated. Conservative substitutions are shown in Table 5 under the heading of “conservative substitutions.” If such substitutions result in a change in biological activity, then more substantial changes, denominated “exemplary substitutions” in Table 3, or as further described below in reference to amino acid classes, may be introduced and the products screened.

TABLE 3
Amino Acid Substitutions

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; |
| | | Phe; Norleucine |
| Leu (L) | Ile | Norleucine; Ile; Val; |
| | | Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile,
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

One type of substitution, for example, that may be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical. Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

The antibodies may also be modified, e.g. in the variable domains of the heavy and/or light chains, e.g., to alter a binding property of the antibody. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. For example, a mutation may be made in one or more of the CDR regions to increase or decrease the K_{D} of the antibody for GUCY2c, to increase or decrease k_{off}, or to alter the binding specificity of the antibody. Techniques in site-directed mutagenesis are well-known in the art. See, e.g., Sambrook et al. and Ausubel et al., supra.

A modification or mutation may also be made in a framework region or constant region to increase the half-life of an anti-GUCY2c antibody. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. In some embodiments, no more than one to five conservative amino acid substitutions are made within the framework region or constant region. In other embodiments, no more than one to three conservative amino acid substitutions are made within the framework region or constant region. According to the invention, a single antibody may have mutations in any one or more of the CDRs or framework regions of the variable domain or in the constant region.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

In a process known as “germlining”, certain amino acids in the VH and VL sequences can be mutated to match those found naturally in germline VH and VL sequences. In particular, the amino acid sequences of the framework regions in the VH and VL sequences can be mutated to match the germline sequences to reduce the risk of immunogenicity when the antibody is administered. Germline DNA sequences for human VH and VL genes are known in the art (see e.g., the “Vbase” human germline sequence database; see also Kabat, E. A., et al., 1991, Sequences of Proteins of Immunological Interest, Fifth Edition, U.S. Department of Health and Human Services, NIH Publication No. 91-3242; Tomlinson et al., 1992, J. Mol. Biol. 227:776-798; and Cox et al., 1994, Eur. J. Immunol. 24:827-836).

Another type of amino acid substitution that may be made is to remove potential proteolytic sites in the antibody. Such sites may occur in a CDR or framework region of a variable domain or in the constant region of an antibody. Substitution of cysteine residues and removal of proteolytic sites may decrease the risk of heterogeneity in the antibody product and thus increase its homogeneity. Another type of amino acid substitution is to eliminate asparagine-glycine pairs, which form potential deamidation sites, by altering one or both of the residues. In another example, the C-terminal lysine of the heavy chain of an anti-GUCY2c antibody of the invention can be cleaved. In various embodiments of the invention, the heavy and light chains of the anti-GUCY2c antibodies may optionally include a signal sequence.

Once DNA fragments encoding the VH and VL segments of the present invention are obtained, these DNA fragments can be further manipulated by standard recombinant DNA techniques, for example to convert the variable region genes to full-length antibody chain genes, to Fab fragment genes, or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be a human IgG₁, IgG₂, IgG₃, IgG₄, IgA, IgE, IgM or IgD, mouse IgA, IgD, IgE, IgG, or IgM, or rabbit IgA, IgE, IgG, or IgM constant region, but most preferably is a rabbit IgG constant region. For a Fab fragment heavy chain gene, the VH-encoding DNA can be operatively linked to another DNA molecule encoding only the heavy chain CH1 constant region. The CH1 heavy chain constant region may be derived from any of the heavy chain genes.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region. The kappa constant region may be any of the various alleles known to occur among different individuals, such as Inv(1), Inv(2), and Inv(3). The lambda constant region may be derived from any of the three lambda genes.

To create a scFv gene, the VH- and VL-encoding DNA fragments are operatively linked to another fragment encoding a flexible linker such that the VH and VL sequences can be expressed as a contiguous single-chain protein, with the VL and VH regions joined by the flexible linker (See e.g., Bird et al., 1988, Science 242:423-426; Huston et al., 1988, Proc. Natl. Acad. Sci. USA 85:5879-5883; McCafferty et al., 1990, Nature 348:552-554. An example of a linking peptide is (GGGGS)₃ (SEQ ID NO: 19), which bridges approximately 3.5 nm between the carboxy terminus of one variable region and the amino terminus of the other variable region. Linkers of other sequences have been designed and used (Bird et al., 1988, supra). Linkers can in turn be modified for additional functions, such as attachment of drugs or attachment to solid supports. The single chain antibody may be monovalent, if only a single VH and VL are used, bivalent, if two VH and VL are used, or polyvalent, if more than two VH and VL are used. Bispecific or polyvalent antibodies may be generated that bind specifically to GUCY2c and to another molecule. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

Other forms of single chain antibodies, such as diabodies, are also encompassed. Diabodies are bivalent, bispecific antibodies in which VH and VL are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., 1993, Proc. Natl. Acad Sci. USA 90:6444-6448; Poljak, R. J., et al., 1994, Structure 2:1121-1123).

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT Publication Nos. WO 91/00360 and WO 92/200373; EP 03089). Heteroconjugate antibodies may be made using any convenient cross-linking methods. Suitable cross-linking agents and techniques are well known in the art, and are described in U.S. Pat. No. 4,676,980.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

The invention also encompasses fusion proteins comprising one or more fragments or regions from the antibodies disclosed herein. In some embodiments, a fusion antibody may be made that comprises all or a portion of an anti-GUCY2c antibody of the invention linked to another polypeptide. In another embodiment, only the variable domains of the anti-GUCY2c antibody are linked to the polypeptide. In another embodiment, the VH domain of an anti-GUCY2c antibody is linked to a first polypeptide, while the VL domain of an anti-GUCY2c antibody is linked to a second polypeptide that associates with the first polypeptide in a manner such that the VH and VL domains can interact with one another to form an antigen binding site. In another preferred embodiment, the VH domain is separated from the VL domain by a linker such that the VH and VL domains can interact with one another. The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

In some embodiments, a fusion polypeptide is provided that comprises at least 10 contiguous amino acids of the variable light chain region shown in SEQ ID NO: 1 and/or at least 10 amino acids of the variable heavy chain region shown in SEQ ID NO: 2. In other embodiments, a fusion polypeptide is provided that comprises at least about 10, at least about 15, at least about 20, at least about 25, or at least about 30 contiguous amino acids of the variable light chain region and/or at least about 10, at least about 15, at least about 20, at least about 25, or at least about 30 contiguous amino acids of the variable heavy chain region. In another embodiment, the fusion polypeptide comprises one or more CDR(s). In still other embodiments, the fusion polypeptide comprises VH CDR3 and/or VL CDR3. For purposes of this invention, a fusion protein contains one or more antibodies and another amino acid sequence to which it is not attached in the native molecule, for example, a heterologous sequence or a homologous sequence from another region. Exemplary heterologous sequences include, but are not limited to a “tag” such as a FLAG tag or a 6His tag. Tags are well known in the art.

A fusion polypeptide can be created by methods known in the art, for example, synthetically or recombinantly. Typically, the fusion proteins of this invention are made by preparing and expressing a polynucleotide encoding them using recombinant methods described herein, although they may also be prepared by other means known in the art, including, for example, chemical synthesis.

In other embodiments, other modified antibodies may be prepared using anti-GUCY2c antibody encoding nucleic acid molecules. For instance, “Kappa bodies” (III et al., 1997, Protein Eng. 10:949-57), “Minibodies” (Martinet al., 1994, EMBO J. 13:5303-9), “Diabodies” (Holliger et al., supra), or “Janusins” (Traunecker et al., 1991, EMBO J. 10:3655-3659 and Traunecker et al., 1992, Int. J. Cancer (Suppl.) 7:51-52) may be prepared using standard molecular biological techniques following the teachings of the specification.

For example, bispecific antibodies, monoclonal antibodies that have binding specificities for at least two different antigens, can be prepared using the antibodies disclosed herein. Methods for making bispecific antibodies are known in the art (see, e.g., Suresh et al., 1986, Methods in Enzymology 121:210). For example, bispecific antibodies or antigen-binding fragments can be produced by fusion of hybridomas or linking of Fab′ fragments. See, e.g., Songsivilai & Lachmann, 1990, Clin. Exp. Immunol. 79:315-321, Kostelny et al., 1992, J. Immunol. 148:1547-1553. Traditionally, the recombinant production of bispecific antibodies was based on the coexpression of two immunoglobulin heavy chain-light chain pairs, with the two heavy chains having different specificities (Millstein and Cuello, 1983, Nature 305, 537-539). In addition, bispecific antibodies may be formed as “diabodies” or “Janusins.” In some embodiments, the bispecific antibody binds to two different epitopes of GUCY2c. In some embodiments, the modified antibodies described above are prepared using one or more of the variable domains or CDR regions from an anti-GUCY2c antibody provided herein.

According to one approach to making bispecific antibodies, antibody variable domains with the desired binding specificities (antibody-antigen combining sites) are fused to immunoglobulin constant region sequences. The fusion preferably is with an immunoglobulin heavy chain constant region, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690.

This invention also provides compositions comprising antibodies conjugated (for example, linked) to an agent that facilitate coupling to a solid support (such as biotin or avidin). For simplicity, reference will be made generally to antibodies with the understanding that these methods apply to any of the GUCY2c binding and/or antagonist embodiments described herein. Conjugation generally refers to linking these components as described herein. The linking (which is generally fixing these components in proximate association at least for administration) can be achieved in any number of ways. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

The antibodies can be bound to many different carriers. Carriers can be active and/or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation. In some embodiments, the carrier comprises a moiety that targets the lung, heart, or heart valve.

An antibody or polypeptide of this invention may be linked to a labeling agent such as a fluorescent molecule, a radioactive molecule or any others labels known in the art. Labels are known in the art which generally provide (either directly or indirectly) a signal.

### Polynucleotides, Vectors, and Host Cells

The invention also provides polynucleotides encoding any of the antibodies, including antibody fragments and modified antibodies described herein, such as, e.g., antibodies having impaired effector function. In another aspect, the invention provides a method of making any of the polynucleotides described herein. Polynucleotides can be made and expressed by procedures known in the art. Accordingly, the invention provides polynucleotides or compositions, including pharmaceutical compositions, comprising polynucleotides, encoding antibody Ab288 or any fragment or part thereof having the ability to antagonize GUCY2c.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a fragment thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a fragment thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene° suite of bioinformatics software (DNASTAR®, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/m1), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). Preferably, the host cells express the cDNAs at a level of about 5 fold higher, more preferably, 10 fold higher, even more preferably, 20 fold higher than that of the corresponding endogenous antibody or protein of interest, if present, in the host cells. Screening the host cells for a specific binding to GUCY2c or a GUCY2c domain is effected by an immunoassay or FACS. A cell overexpressing the antibody or protein of interest can be identified.

An expression vector can be used to direct expression of an anti-GUCY2c antibody. One skilled in the art is familiar with administration of expression vectors to obtain expression of an exogenous protein in vivo. See, e.g., U.S. Pat. Nos. 6,436,908; 6,413,942; and 6,376,471. Administration of expression vectors includes local or systemic administration, including injection, oral administration, particle gun or catheterized administration, and topical administration. In another embodiment, the expression vector is administered directly to the sympathetic trunk or ganglion, or into a coronary artery, atrium, ventrical, or pericardium.

Targeted delivery of therapeutic compositions containing an expression vector, or subgenomic polynucleotides can also be used. Receptor-mediated DNA delivery techniques are described in, for example, Findeis et al., Trends Biotechnol., 1993, 11:202; Chiou et al., Gene Therapeutics: Methods And Applications Of Direct Gene Transfer, J. A. Wolff, ed., 1994; Wu et al., J. Biol. Chem., 1988, 263:621; Wu et al., J. Biol. Chem., 1994, 269:542; Zenke et al., Proc. Natl. Acad. Sci. USA, 1990, 87:3655; Wu et al., J. Biol. Chem., 1991, 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. The therapeutic polynucleotides and polypeptides can be delivered using gene delivery vehicles. The gene delivery vehicle can be of viral or non-viral origin (see generally, Jolly, Cancer Gene Therapy, 1994, 1:51; Kimura, Human Gene Therapy, 1994, 5:845; Connelly, Human Gene Therapy, 1995, 1:185; and Kaplitt, Nature Genetics, 1994, 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5, 219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther., 1992, 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther., 1992, 3:147); ligand-linked DNA (see, e.g., Wu, J. Biol. Chem., 1989, 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell Biol., 1994, 14:2411, and in Woffendin, Proc. Natl. Acad. Sci., 1994, 91:1581.

### Compositions

The invention also provides compositions comprising an effective amount of an anti-GUCY2c antibody described herein. Examples of such compositions, as well as how to formulate, are also described herein. In some embodiments, the composition comprises one or more GUCY2c antibodies. In other embodiments, the anti-GUCY2c antibody recognizes GUCY2c. In other embodiments, the anti-GUCY2c antibody is a mouse antibody. In other embodiments, the anti-GUCY2c antibody is a rabbit chimeric or rabbitized antibody.

It is understood that the compositions can comprise more than one anti-GUCY2c antibody (e.g., a mixture of GUCY2c antibodies that recognize different epitopes of GUCY2c). Other exemplary compositions comprise more than one anti-GUCY2c antibody that recognize the same epitope(s), or different species of anti-GUCY2c antibodies that bind to different epitopes of GUCY2c. In some embodiments, the compositions comprise a mixture of anti-GUCY2c antibodies that recognize different variants of GUCY2c.

The composition used in the present invention can further comprise pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The anti-GUCY2c antibody and compositions thereof can also be used in conjunction with, or administered separately, simultaneously, or sequentially with other agents that serve to enhance and/or complement the effectiveness of the agents.

The invention also provides compositions, including pharmaceutical compositions, comprising any of the polynucleotides of the invention. In some embodiments, the composition comprises an expression vector comprising a polynucleotide encoding the antibody as described herein. In other embodiment, the composition comprises an expression vector comprising a polynucleotide encoding any of the antibodies described herein.

### Methods for Diagnoising or Treating Conditions Mediated by GUCY2c

The antibodies and the antibody conjugates of the present invention are useful in various applications including, but are not limited to, diagnostic treatment methods and therapeutic treatment methods.

In one aspect, provided is a method of detecting, diagnosing, and/or monitoring a cancer. For example, the GUCY2c antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent. The anti-GUCY2c antibodies described herein can be used, for example without limitation, for immunohistochemical staining, Western blot analysis, and/or assay of sample fluids to detect presence of GUCY2c. GUCY2c expression may be determined in a diagnostic or prognostic assay by evaluating increased levels of GUCY2c present in a sample—e.g., via an immunohistochemistry assay using the anti-GUCY2c antibodies described herein.

In another aspect, the invention provides a method for treating a cancer. In some embodiments, the method of treating a cancer in a subject comprises administering to the subject in need thereof an effective amount of a composition (e.g., pharmaceutical composition) comprising any of the GUCY2c antibodies as described herein. As used herein, cancers include, but are not limited to colorectal cancer, bladder cancer, breast cancer, cervical cancer, choriocarcinoma, colon cancer, esophageal cancer, gastric cancer, glioblastoma, glioma, brain tumor, head and neck cancer, kidney cancer, lung cancer, oral cancer, ovarian cancer, pancreatic cancer, prostate cancer, liver cancer, uterine cancer, bone cancer, leukemia, lymphoma, sacrcoma, blood cancer, thyroid cancer, thymic cancer, eye cancer, and skin cancer. In some embodiments, the cancer is gastric cancer. In some embodiments, provided is a method of inhibiting tumor growth or progression in a subject, comprising administering to the subject in need thereof an effective amount of a composition comprising the GUCY2c antibodies or the GUCY2c antibody conjugates as described herein. In some embodiments, the tumor is a GUCY2c expressing tumor. In other embodiments, provided is a method of inhibiting metastasis of cancer cells in a subject, comprising administering to the subject in need thereof an effective amount of a composition comprising any of the GUCY2c antibodies as described herein. In other embodiments, provided is a method of inducing regression of a tumor in a subject, comprising administering to the subject in need thereof an effective amount of a composition comprising any of the GUCY2c antibodies as described herein.

In embodiments that refer to a method of diagnosis or treatment as described herein, such embodiments are also further embodiments for use in that method of diagnosis or treatment, or alternatively for the manufacture of a medicament for use in that treatment.

In another aspect, the invention provides an anti-GUCY2c antibody as described herein for use in therapy. The invention further provides the use of an anti-GUCY2c antibody as described herein in the manufacture of a medicament for use in therapy. In some embodiments, the therapy is a method of treating of a cancer in a subject. In some embodiments, the therapy is a method of inhibiting tumor growth or progression in a subject; inhibiting metastasis of cancer cells in a subject; or inducing regression of a tumor in a subject.

With respect to all methods described herein, reference to anti-GUCY2c antibodies also includes compositions comprising one or more additional agents. These compositions may further comprise suitable excipients, such as pharmaceutically acceptable excipients including buffers, which are well known in the art. The present invention can be used alone or in combination with other methods of treatment.

In some embodiments, an anti-GUCY2c antibody is used in conjunction with one or more other diagnostic antibodies, such as, for example without limitation, an antibody targeting PD-L1, CD19, CD22, CD40, CD52, or CCR4.

### Formulations

Formulations of the anti-GUCY2c antibody used in accordance with the present invention are prepared for storage by mixing an antibody having the desired degree of purity with optional carriers, excipients or stabilizers (Remington, The Science and Practice of Pharmacy 20th Ed. Mack Publishing, 2000), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

### Kits

The invention also provides kits comprising any or all of the antibodies described herein. Kits of the invention include one or more containers comprising an anti-GUCY2c antibody described herein and instructions for use in accordance with any of the methods of the invention described herein. Generally, these instructions comprise a description of use of the anti-GUCY2c antibody for the above described diagnostic or therapeutic treatments. In some embodiments, a kit can contain both a first container having a dried protein and a second container having an aqueous formulation.

In some embodiments, the antibody is a rabbit chimeric antibody. In some embodiments, the antibody is a rabbitized antibody. In some embodiments, the antibody is a monoclonal antibody. The instructions relating to the use of an anti-GUCY2c antibody generally include information as to the use for detecting presence of GUCY2c in a sample, such as for example by immunohistochemistry. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like.

Kits may optionally provide additional components such as buffers and interpretive information. Normally, the kit comprises a container and a label or package insert(s) on or associated with the container.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLE

### Example 1

### Anti-GUCY2c Antibodies

This Example illustrates the identification of anti-GUCY2c antibodies suitable for detecting GUCY2c in a sample.

Fifty six hybridoma supernatants from BALB/c mice immunized against GUCY2c were tested for immunoreactivity in formalin fixed paraffin embedded cell pellets. Cell pellets generated for this screen consisted of 300.19 parental cells that do not express GUCY2c, and 300.19 cells over-expressing mouse, cynomolgus macaque or human GUCY2c, the T84 human colorectal cancer cell line expressing endogenous GUCY2c, and the HT29 human colorectal cancer cell line that is negative for GUCY2c expression. Cell lines were fixed for 24 h in 10% neutral buffered formalin (Thermo Scientific) and centrifuged at 300×g for 4 minutes (m) to pellet. Formalin was removed and cells pellets were re-suspended gently with pre-warmed 50° C. Histogel (Thermo Scientific). Cell pellets embedded in Histogel were cooled at 4° C. for 1-2 h before being processed overnight in a VIP automated tissue processor (Tissue-Tek). Processed cell pellets were embedded in paraffin. A cell microarray containing a core of each of the cell lines above was generated as follows: Each donor block containing a cell pellet was cored with a 2 mm biopsy punch (Miltex) and placed in a 2 mm hole in a recipient block generated from a 72 core rubber array mold (ARRAYMOLD). Empty holes were filled with paraffin and the array was allowed to warm at 40 ° C. in an incubator overnight to anneal the donor cores with the recipient block. Five micron sections of the cell microarray were cut, transferred to a water bath and placed on Superfrost Excell microscope slides (Fisher). Slides were allowed to dry overnight. After deparaffinization and rehydration of tissue sections, heat induced epitope retrieval was performed in the Retriever 2100 pressure cooker (Electron Microscopy Sciences) in Borg Decloaker buffer pH 9.5 (Biocare Medical) or pH 6.0 Citrate buffer (Thermo Scientific) followed by cooling to room temperature (RT). Endogenous peroxidase activity was inactivated with Peroxidazed 1 (Biocare Medical) for 10 m. Non-specific protein interactions were blocked for 10 m with Background Punisher (Biocare Medical). Each hybridoma was incubated without dilution for 1 h under both heat induced epitope retrieval conditions. Sections were rinsed in TBS and hybridoma binding was detected with Envision+ Mouse HRP (DAKO) for 30 m. Slides were rinsed in TBS and immunoreactivity was developed with Betazoid DAB Chromogen Kit (Biocare Medical) for 5 m, followed by rinses in distilled water. Immunostained sections were briefly counterstained with CAT Hematoxylin (Biocare Medical), washed in tap water, dehydrated in graded alcohols, cleared in xylene, and coverslipped with Permount mounting medium (FisherChemicals). Slides were evaluated by a pathologist to assess immunoreactivity.

Six of the hybridoma supernatants were affinity purified and submitted for immunohistochemical testing. The purified mouse IgG clones were tested for immunoreactivity on freshly cut sections from the same cell pellets used for the original hybridoma screen. All steps for the immunohistochemistry method were the same as the hybridoma supernatant screen except that the purified IgGs were tested at 2 μg/ml and 10 μg/ml. Results are summarized in Table 4 below. In Table 4, a plus sign (+) indicates that the staining with the indicated antibody was detected, and a minus sign (−) indicates lack of staining.

TABLE 4

| | Staining in | Staining in | Staining in | Staining in |
|---|---|---|---|---|
| | cells with | cells over- | cells over- | cells over- |
| | endogenous | expressing | expressing | expressing |
| Antibody | GUCY2c | human | cyno | mouse |
| name | expression | GUCY2c | GUCY2c | GUCY2c |
| A | + | + | + | + |
| B | + | + | + | − |
| C | + | + | + | + |
| D | + | + | + | + |
| Ab288 | + | + | + | − |
| E | + | + | − | − |

Anti-GUCY2c antibody Ab288 detected GUCY2c in cells that naturally express GUCY2c, cells that express transgenic human GUCY2c, and cells that express transgenic cynomolgus monkey GUCY2c (Table 4). Anti-GUCY2c antibody Ab288 did not stain cells expressing mouse GUCY2c. Furthermore, both membrane staining and cytoplasmic staining of human and cynomolgus monkey tissue were observed with anti-GUCY2c antibody Ab288.

These results demonstrate that anti-GUCY2c antibody Ab288 specifically binds to human and cynomolgus monkey GUCY2c and does not crossreact with mouse GUCY2c. Anti-GUCY2c antibody Ab288 can detect GUCY2c on the membrane and in the cytoplasm.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. An isolated antibody that specifically binds to Guanylyl cyclase C (GUCY2c) and comprises:
a heavy chain variable region (VH) comprising a VH complementarity determining region one (CDR1), VH CDR2, and VH CDR3 of the amino acid sequence shown in SEQ ID NO: 2.
a light chain variable region (VL) comprising a VL CDR1, VL CDR2, and VL CDR3 of the amino acid sequence shown in SEQ ID NO: 1.

2. The isolated antibody of claim 1, wherein the antibody comprises a VH CDR1 comprising the amino acid sequence of SEQ ID NO: 6, a VH CDR2 comprising the amino acid sequence of SEQ ID NO: 7, a VH CDR3 comprising the amino acid sequence shown in SEQ ID NO: 8, a VL CDR1 comprising the amino acid sequence shown in SEQ ID NO: 3, a VL CDR2 comprising the amino acid sequence shown in SEQ ID NO: 4 and a VL CDR3 comprising the amino acid sequence shown in SEQ ID NO: 5.

3. The isolated antibody of claim 1, wherein the antibody comprises a VH comprising the amino acid sequence shown in SEQ ID NO: 2, or a variant thereof with one or several conservative amino acid substitutions in residues that are not within a CDR.

4. The isolated antibody of claim 3, wherein the antibody comprises a VL comprising the amino acid sequence shown in SEQ ID NO: 1, or a variant thereof with one or several amino acid substitutions in amino acids that are not within a CDR.

5. The isolated antibody of claim 2, wherein the antibody comprises a VH comprising the amino acid sequence shown in SEQ ID NO: 2, and a VL comprising the amino acid sequence shown in SEQ ID NO: 1.

6. The isolated antibody of claim 2, wherein the antibody comprises a heavy chain comprising the amino acid sequence shown in SEQ ID NO: 9, 10, 12 or 13 and a light chain comprising the amino acid sequence shown in SEQ ID NO: 11, 14, 15, 16, 17, 18, or 19.

7. The isolated antibody of claim 6, wherein the antibody comprises a heavy chain comprising the amino acid sequence shown in SEQ ID NO: 9 or 10 and a light chain comprising the amino acid sequence shown in SEQ ID NO: 11.

8. The isolated antibody of claim 6, wherein the antibody comprises a heavy chain comprising the amino acid sequence shown in SEQ ID NO: 12 or 13 and a light chain comprising the amino acid sequence shown in SEQ ID NO: 14, 15, 16, 17, 18, or 19.

9. The isolated antibody of claim 2, wherein the antibody comprises a constant region.

10. The isolated antibody of claim 9, wherein the antibody has an isotype that is selected from the group consisting of mouse IgG₁ or rabbit IgG.

11. The isolated antibody of claim 9, wherein the antibody is a rabbit chimeric antibody or a rabbitized antibody.

12. (canceled)

13. The isolated antibody of claim 2, wherein the antibody specifically binds to human and cynomolgus monkey GUCY2c.

14. The isolated antibody of claim 13, wherein the antibody does not bind to mouse GUCY2c.

15. The isolated antibody of claim 2, wherein the antibody binds to cytoplasmic GUCY2c.

16. An isolated cell line that produces the antibody of claim 2.

17. An isolated nucleic acid encoding the antibody of claim 2.

18. A recombinant expression vector comprising the nucleic acid of claim 17.

19. A host cell comprising the expression vector of claim 18.

20. A hybridoma capable of producing the antibody of claim 2.

21. (canceled)

22. (canceled)

23. (canceled)

24. (canceled)

25. A composition comprising the antibody of claim 2, and a carrier.

26. A kit for the diagnosis of cancer comprising the composition of claim 25.

27. A method for diagnosing cancer in a subject, the method comprising contacting a test sample of tissue cells suspected of containing cancerous tumor cells obtained from the subject with the antibody of claim 2.

28. The method of claim 27, further comprising detecting the formation of a complex between the antibody and GUCY2c in the sample, and classifying a higher level of formation of such a complex in the test sample as compared to the level of formation of such a complex in a control sample of normal tissue cells from the same type of tissue as the sample, as diagnostic of the presence of cancer in the subject.

29. The method of claim 27, wherein the cancer is selected from the group consisting of colorectal cancer, gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma.

30. The method of claim 27, wherein the antibody is detectably labeled.

31. A method of diagnosing the presence of cancer in a subject, the method comprising
determining the level of expression of Guanylyl cyclase C (GUCY2c) in
a test sample of tissue cells obtained from tissue suspected of containing cancerous tumor cells in the subject and
a control sample of known normal cells obtained from the same type of tissue as the test sample,
wherein determining the level of expression of GUCY2c comprises employing an antibody of claim 2, and
classifying a higher level of expression of GUCY2c in the test sample as compared to the control sample, as diagnostic of the presence of cancer in the subject from which the test sample was obtained.

32. The method of claim 31, wherein the step employing the antibody comprises an immunohistochemistry or Western blot analysis.


SEQUENCE LISTING (19 sequences)

SEQ ID NO: 1 - protein, 111 aa, Mus musculus
DIVLTQSPASLAVSLGQRATISCRASESVEYFGTSFMQWYQQRPGQPPKLLIYAASNVESGVPVRFSGSGSGTDFSLNIHPVEEDDIAMYFCQQSRKVPWTFGGGTNLEIK

SEQ ID NO: 2 - protein, 118 aa, Mus musculus
DVQLQESGPGLVKPSQSLSLTCTVTGYSITSDYAWNWIRQFPGNNLEWMGYISYSGSTRYNPSLKSRISITRDTSKNQFFLQLNSVTSEDTATYYCAREDGYVAMDYWGQGTSVTVSS

SEQ ID NO: 3 - protein, 15 aa, Mus musculus
RASESVEYFGTSFMQ

SEQ ID NO: 4 - protein, 7 aa, Mus musculus
AASNVES

SEQ ID NO: 5 - protein, 9 aa, Mus musculus
QQSRKVPWT

SEQ ID NO: 6 - protein, 6 aa, Mus musculus
SDYAWN

SEQ ID NO: 7 - protein, 16 aa, Mus musculus
YISYSGSTRYNPSLKS

SEQ ID NO: 8 - protein, 9 aa, Mus musculus
EDGYVAMDY

SEQ ID NO: 9 - protein, 442 aa, Mus musculus
DVQLQESGPGLVKPSQSLSLTCTVTGYSITSDYAWNWIRQFPGNNLEWMGYISYSGSTRYNPSLKSRISITRDTSKNQFFLQLNSVTSEDTATYYCAREDGYVAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 10 - protein, 441 aa, Mus musculus
DVQLQESGPGLVKPSQSLSLTCTVTGYSITSDYAWNWIRQFPGNNLEWMGYISYSGSTRYNPSLKSRISITRDTSKNQFFLQLNSVTSEDTATYYCAREDGYVAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 11 - protein, 218 aa, Mus musculus
DIVLTQSPASLAVSLGQRATISCRASESVEYFGTSFMQWYQQRPGQPPKLLIYAASNVESGVPVRFSGSGSGTDFSLNIHPVEEDDIAMYFCQQSRKVPWTFGGGTNLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 441 aa, Artificial Sequence
  Chimeric rabbit sequence
DVQLQESGPGLVKPSQSLSLTCTVTGYSITSDYAWNWIRQFPGNNLEWMGYISYSGSTRYNPSLKSRISITRDTSKNQFFLQLNSVTSEDTATYYCAREDGYVAMDYWGQGTSVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 13 - protein, 440 aa, Artificial Sequence
  chimeric rabbit sequence
DVQLQESGPGLVKPSQSLSLTCTVTGYSITSDYAWNWIRQFPGNNLEWMGYISYSGSTRYNPSLKSRISITRDTSKNQFFLQLNSVTSEDTATYYCAREDGYVAMDYWGQGTSVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPG

SEQ ID NO: 14 - protein, 215 aa, Artificial Sequence
  chimeric rabbit sequence
DIVLTQSPASLAVSLGQRATISCRASESVEYFGTSFMQWYQQRPGQPPKLLIYAASNVESGVPVRFSGSGSGTDFSLNIHPVEEDDIAMYFCQQSRKVPWTFGGGTNLEIKGDPVAPTVLIFPPAADQVATGTVTIVCVANKYFPDVTVTWEVDGTTQTTGIENSKTPQNSADCTYNLSSTLTLTSTQYNSHKEYTCRVTQGTTSVVQSFNRGDC

SEQ ID NO: 15 - protein, 215 aa, Artificial Sequence
  chimeric rabbit sequence
  VARIANT (174)..(174): Xaa is Ala, Asp, Glu, Phe, Gly, His, Ile, Lys, Leu, Met, Asn, Pro, Gln, Arg, Ser, Thr, Val, Trp, or Tyr
DIVLTQSPASLAVSLGQRATISCRASESVEYFGTSFMQWYQQRPGQPPKLLIYAASNVESGVPVRFSGSGSGTDFSLNIHPVEEDDIAMYFCQQSRKVPWTFGGGTNLEIKGDPVAPTVLIFPPAADQVATGTVTIVCVANKYFPDVTVTWEVDGTTQTTGIENSKTPQNSADXTYNLSSTLTLTSTQYNSHKEYTCRVTQGTTSVVQSFNRGDC

SEQ ID NO: 16 - protein, 215 aa, Artificial Sequence
  chimeric rabbit sequence
DIVLTQSPASLAVSLGQRATISCRASESVEYFGTSFMQWYQQRPGQPPKLLIYAASNVESGVPVRFSGSGSGTDFSLNIHPVEEDDIAMYFCQQSRKVPWTFGGGTNLEIKGDPVAPTVLIFPPAADQVATGTVTIVCVANKYFPDVTVTWEVDGTTQTTGIENSKTPQNSADSTYNLSSTLTLTSTQYNSHKEYTCRVTQGTTSVVQSFNRGDC

SEQ ID NO: 17 - protein, 215 aa, Artificial Sequence
  chimeric rabbit sequence
DIVLTQSPASLAVSLGQRATISCRASESVEYFGTSFMQWYQQRPGQPPKLLIYAASNVESGVPVRFSGSGSGTDFSLNIHPVEEDDIAMYFCQQSRKVPWTFGGGTNLEIKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 18 - protein, 215 aa, Artificial Sequence
  chimeric rabbit sequence
  VARIANT (174)..(174): Xaa is Ala, Asp, Glu, Phe, Gly, His, Ile, Lys, Leu, Met, Asn, Pro, Gln, Arg, Ser, Thr, Val, Trp, or Tyr
DIVLTQSPASLAVSLGQRATISCRASESVEYFGTSFMQWYQQRPGQPPKLLIYAASNVESGVPVRFSGSGSGTDFSLNIHPVEEDDIAMYFCQQSRKVPWTFGGGTNLEIKGDPVAPTVLIFPPAADQVATGTVTIVCVANKYFPDVTVTWEVDGTTQTTGIENSKTPQNSADXTYNLSSTLTLTSTQYNSHKEYTCKVTQGTTSVVQSFNRGDC

SEQ ID NO: 19 - protein, 215 aa, Artificial Sequence
  chimeric rabbit sequence
DIVLTQSPASLAVSLGQRATISCRASESVEYFGTSFMQWYQQRPGQPPKLLIYAASNVESGVPVRFSGSGSGTDFSLNIHPVEEDDIAMYFCQQSRKVPWTFGGGTNLEIKGDPVAPTVLIFPPAADQVATGTVTIVCVANKYFPDVTVTWEVDGTTQTTGIENSKTPQNSADSTYNLSSTLTLTSTQYNSHKEYTCKVTQGTTSVVQSFNRGDC